(19) United States
(12) Patent Application Publication
(10) Pub. No.: US 2008/0243173 A1
(43) Pub. Date: Oct. 2, 2008

(54) Bi-Directional Tourniquet

(76) Inventor: Patricia Thorpe, Iowa City, IA (US)

Correspondence Address: BULLIVANT HOUSER BAILEY PC, 1415 L STREET, SUITE 1000, SACRAMENTO, CA, 95814, US

(21) Appl. No.: 11/955,806

(22) Filed: Dec. 13, 2007

Related U.S. Application Data
(63) Continuation-in-part of application No. 11/940,225, filed on Nov. 14, 2007, now abandoned.
(60) Provisional application No. 60/901,715, filed on Feb. 13, 2007.
(60) Provisional application No. 60/875,087, filed on Dec. 13, 2006.

(51) Int. Cl. A61B 17/00

(52) U.S. Cl. 606/203

(57) ABSTRACT

A tourniquet comprising a band comprised of an elastic and flexible material, the band having a width and a length, a first end and a second end, and a first face and second face, where at least a portion of the first face comprises hook-type fastener means located within a third of the band at the first end, within a third of the band at the second end, and within a middle third of the band, and where the second face comprises loop-type fastener means spanning the length of the second face.

### PRIORITY

This invention is a continuation-in-part of U.S. patent application Ser. No. 11/940,225, filed Nov. 14, 2007, which is a continuation-in-part of U.S. Provisional Patent Application No. 60/901,715, filed Feb. 13, 2007, and a continuation-in-part of U.S. Provisional Patent Application No. 60/875,087, filed Dec. 13, 2006, the full disclosure of each of which applications are incorporated herein in their entirety.

### BACKGROUND OF THE INVENTION

1. Field of the Invention

This invention describes a versatile trauma tourniquet with features permitting ease of application in the field, including hook and loop along target securement areas.

2. Description of Related Art

Wartime trauma from exploding devices and gunshot wounds results in a high incidence of life and limb-threatening injuries. Exsanguination from peripheral vascular injury is the primary preventable cause of death in field trauma, and has been well documented in literature regarding current war trauma.

Blood loss from non-fatal injuries to arms and legs also can contribute to morbidity to kidneys, liver and other vital organs due to hypotension. The loss of significant amounts of blood also increases the rate of consumption of blood products from the blood bank. Multiple extremity wounds can also complicate triage.

It is axiomatic that in combat, there is a “platinum 5 minutes” for major vascular injury. Even with quick triage, it often takes hours to transport casualties off the battlefield, and even if the distance is small, the hazardous nature of the forward combat areas frequently prevents medical personnel from quickly reaching the wounded. Hence the need for individual soldiers to have a readily available tourniquet that is safe and effective.

Thus, despite the advances in modern medical care for the battlefield, control of blood loss is the one immediate treatment that can be applied to or by an injured soldier to decrease the chance of organ injury, limb loss and death due to hemorrhage. While deep vein thrombosis is of concern to soldiers and doctors in the field, the more immediate danger is always focal vascular injury and uncontrolled hemorrhage.

Despite long-established use in the medical field, the need for new tourniquet technology is readily acknowledged. Hemorrhage from extremity wounds is the leading cause of preventable death on the battlefield, and tourniquets are the most viable option for controlling life-threatening extremity hemorrhage in the tactical phase of an operation. The evolution of tourniquet technology has been a marked response to the realities of fighting in Iraq and Afghanistan.

Tourniquet designs are varied. One recent patent, U.S. Pat. No. 6,189,538, describes a non-pneumatic tourniquet for use in treating deep vein thrombosis that includes a band having a first end and a second end, wherein the first end and second end have a structure for adjustably connecting to one another, shown as hook and loop fasteners, and an adjustable disc made of a substantially hard, non-compressible material connected to the band. Provided is a method of treating deep vein thrombosis in which the thrombus cannot be easily treated using a catheter.

United States Patent Application No. 2007/0005107 is directed to a military emergency tourniquet, described as a tourniquet for rapidly and easily reducing or stopping blood flow to a limb. The tourniquet utilizes a hook and loop system and includes a twistable strap, a base including two opposing entry apertures and an exit aperture, a windlass and at least one receiving loop.

U.S. Pat. No. 4,182,338, provides a pressure applying device which prevents bleeding through needle puncture wounds by applying pressure to the wound through an elastomeric appliance having a blunt skin abutting surface held in place over the wound by securing straps. The pressure applied by the device is sufficient only to prevent bleeding through the wound and is not designed to impede the subsurface flow of blood.

The most commonly used tourniquet, however, is still a length of surgical tubing, the tourniquet used in phlebotomy. However, such tubing does not function well for greater than 1-2 minutes because of the pain.

A continuing problem in the field, then, is the need for rapid and safe protection for injuries to extremities. Vascular injury alone can result in amputations from blood loss and resulting ischemic muscle. The existing tourniquets available to the combat infantryman, as well as medical personnel, vary in degree of mechanical/clinical failure, often due to complicated and time consuming application. In addition, certain designs are painful, either inadequately or too adequately diminish blood-flow, or are too bulky for routine field use.

Ease of use and effectiveness of the application are critical to saving lives and limbs during those platinum 5 minutes, yet according to the U.S. Army Institute of Surgical Research, current technology has reportedly fostered misapplications, intolerable pinching and skin/tissue damage, and has yet to take effective control of leg injuries. Studies have shown that tourniquet failure has revolved around several issues: 1) inadequate mechanical advantage for tightening, 2) device failure (i.e., breakage), and 3) intolerable pinching or circumferential pain prior to pulse elimination. While one-handed tourniquet technologies have even been able to minimize blood flow in the arm to some extent, no one-handed technology has been found to be successful in easily and reliably decreasing blood flow to the lower extremity.

In April 2007, a comparative report from The Naval Sea Systems Command was released to the public. This comprehensive review of available field tourniquets compares 13 designs from 12 manufacturers. The comparison was conducted under rigorous clinical standards, and the comments from failed applications provide great insight into the application realities of tourniquet designs. They also demonstrated the need for versatility and immediate availability of a simple, strong and effective tourniquet in the field.

There remains, then, a need for a life and limb saving tourniquet, for military use and civilian use that can overcome the obstacles of current technology.

### SUMMARY OF THE INVENTION

This invention solves these problems with a vascular tourniquet that is lightweight, not bulky, simple, fast and effective to apply in a battle setting or civilian emergency situations.

In one embodiment, the tourniquet comprises a band of elastic and flexible material, having a width and a length, a first end and a second end, and a first face and second lace, where at least a portion of the first face comprises hook-type fastener means located within a third of the band at the first end, within a third of the band at the second end, and within a middle third of the band, and where the second face comprises loop-type fastener means spanning the length of the second face.

In one preferred aspect, the band is applied across a body party, such as an appendage, head, neck or torso, the band being adapted to encircle the body part bi-directionally. As the band is extended from its first end to encircle the body part, the loop-type fastener means of the second face engages the hook-type fastener means of the first face, which may be at the second end, where the body part has a large diameter, or, more typically, at the middle third of the tourniquet.

Where the band has engaged at the middle third, the tourniquet is further extended from its second end to encircle the body part, and the loop-type fastener means of the second face engage the hook-type fastener means of the first face at the first end previously attached over the middle portion. The pressure potential of the tourniquet to curtail bleeding is significantly increased with the additional hook and loop attachment where the application of the tourniquet comprises the loop-type fastener means of the second face at the first end being engaged with the hook-type fastener means of the middle third of the first face, and the loop-type fastener means of the second face of the second end being engaged with the hook-type fastener means of the first face at the first end.

In an alternative embodiment of the invention, a point of attachment is provided for the tourniquet on an article of clothing, or garment, such as through providing hook-type fastener material secured to the garment.

In a different embodiment, the tourniquet of the invention may include use of a movable portable disc, which can be selectively secured to the tourniquet and situated to apply focal pressure at a specific location, such as a location of hemorrhage or sub-surface blood flow.

With the tourniquet, a method for impeding blood flow to a body part can be utilized by applying the tourniquet to compress a vessel at a region to stop bleeding and also may partially decrease blood flow to tissue supplied below that region. The method comprises applying a tourniquet comprising a band comprised of an elastic and flexible material, the band having a width and a length, a first end and a second end, and a first face and second face, where at least a portion of the first face comprises hook-type fastener means located within a third of the band at the first end, within a third of the band at the second end, and within a middle third of the band, and where the second face comprises loop-type fastener means spanning the length of the second face, whereby the band is adapted to encircle the body part bidirectionally. The tourniquet is applied at a specific region by extending the band from its first end to encircle the body part, whereby the loop-type fastener means of the second face engages the hook-type fastener means of the first face and subsequently extending the band from its second end to encircle the body part, in the opposite direction, whereby the loop-type fastener means of the second face engage the hook-type fastener means of the first face, securely enough that blood flow is decreased to tissue below the region.

In a preferred such method the loop-type fastener means of the second face of the tourniquet at the first end engages the hook-type fastener means of the middle third of the first face, and then the loop-type fastener means of the second face at the second end engages the hook-type fastener means of the first face at the first end.

The tourniquet can also be sized so that it may go around any part of the body that may be treatable by the application of pressure to reduce hemorrhage or sub-surface blood flow, including, but not limited to, the neck, head and torso, as well as upper and lower extremities.

A given tourniquet may optionally be applied to a larger body part than it was originally intended, by applying the tourniquet so that the loop-type fastener means of the second face at the first end engages the hook-type fastener means of the first face at the second end, by-passing the middle third.

The methods of the invention permit various levels of pressure, due to the elastic band, and the blood flow decrease may be partial to substantially complete. The method of the invention can be applied to decrease bleeding at the site and the distal bloodflow as determined by Doppler flow measurements or pulse palpitation.

These and other features and advantages of this invention are described in, or are apparent from, the following detailed description of various exemplary embodiments of the apparatus and methods according to this invention.

### BRIEF DESCRIPTION OF THE DRAWINGS

FIG. 1 shows a plan view of the tourniquet, with the second face, showing the loop fastener material of a hook and loop fastener system.

FIG. 2 shows a plan view of the first face of the tourniquet, having hook-type fastener material facing up.

FIG. 3 is a depiction of the tourniquet applied over a limb with the loop fastener end against the limb, and the hook fastener regions facing up.

FIG. 4 depicts the further application of the tourniquet of FIG. 3, engaging the limb with both ends wrapped beneath the limb.

FIG. 5 shows the tourniquet of FIG. 4, with a first end engaging the first surface of the tourniquet and the loop fastener of the first end engaging the hook fastener region of the middle third of the tourniquet.

FIG. 6 shows the final application of the tourniquet shown previously in FIG. 5, with the second end engaging the first surface of the tourniquet with the loop fastener of the second end engaging the hook type fastener region of the first end of the tourniquet.

### DETAILED DESCRIPTION OF THE INVENTION

The products and methods of the present invention provide a versatile tourniquet that can be used in the field by military personnel, or in the clinical setting by medical personnel, or in a non-combat situation where trauma hemorrhage is an issue.

In this description, by the term reversible fastening means is meant any means, such as loop and hook fastener that allows secure fastening of the tourniquet components, but that can be simply and manually reversibly detached.

By tourniquet is meant any means for impeding blood flow or blood loss at a site by compressing a vessel or series of vessels by applying the strap, tube or other means at a region to selectively decrease blood flow and stop bleeding.

By bi-directional is meant that the tourniquet is supplied with two free ends, and can be applied by placing the center against the extremity and pulling the first free end to secure it against the center, and then pulling the opposite free end in the opposite direction, such as to secure the tourniquet over the extremity with resistive force from the elastic applied within the tourniquet in both directions.

The tourniquet can be applied by medical personnel, such as a trained physician, nurse, or emergency technician, as well as an injured soldier, whether medically trained or a fellow soldier, in less than 30 seconds.

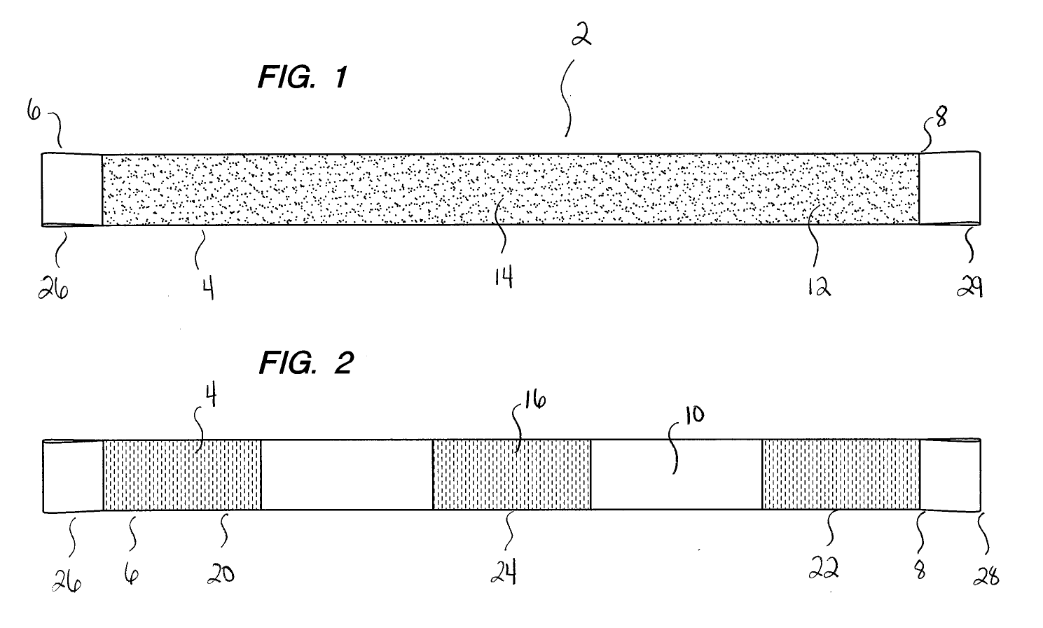

Looking to FIGS. 1 and 2, the tourniquet 2 comprises a band 4 of elastic and flexible material, having a width and a length, a first end 6 and a second end 8, and a first face 10 and second face 12. The width of the band 4 is desirably on the order of about 1½ to 2 inches. Fabric thickness can vary, depending on the materials, but is on the order of about 1/12 inch, down to about 1/16 inch or less, for an appropriate fabric having the strength and elasticity for the overall comfort and blood flow restriction requirements of the tourniquet 2.

The length of the tourniquet 2 may vary, and multiple tourniquets 2 can be supplied for varying applications. Where the appendage 30 is an arm, desirable tourniquet dimensions are from about a minimum of 24 inches to a maximum of about 30 inches.

For a leg, the dimensions are on the average order of about 30 inches minimum to a maximum of about 40 inches.

Tourniquet 2 can also be supplied in varying sizes, for instance in sizes of small, medium, large and extra large, having lengths of, respectively, 28 inches, 32 inches, 36 inches and 38 inches, to provide one example. Larger sizes may be required, depending on the size and fitness of the intended populace.

A pediatric version of the tourniquet 2 can be provided with smaller dimensions configured to be appropriate for children.

In reference to FIG. 1, the second face comprises one component of a reversible fastening means, such as a loop-type fastener means 14 spanning the length of the second face.

As best seen in reference to FIG. 2, at least a portion of the first face comprises a complementary reversible fastener means, such as a hook-type fastener means 16. These can be located at discrete areas as attached segments of hook-type fastener means. At a minimum, the hook-type fastener should be present at a third of the band at the first end 20, within a third of the band at the second end 22, and within a middle third, or middle portion 24, of the band. Portions 20, 22 and 24 can vary in size, from a minimum of about 2 inches up to about 9 inches or longer. The hoop-type fastener sections 20, 22 and 24 can be secured by any conventional means known to the art, such as machine stitched to the first face 10 of the band 4 at designated intervals. The quality of the reversible fastener system employed should be such that the first face 10 of the tourniquet 2 strongly adheres to the second face 12. The hoop-type fastener sections can also be applied by stapling or gluing, or the like, to the band 4. The overall coverage of the hook-type fastener material 16 should leave open substantial regions of the band 4, to allow bidirectional stretch of the band 4.

Pull tabs can be supplied 26 and 28 at the first 6 and second ends 8, respectively, of the tourniquet 2.

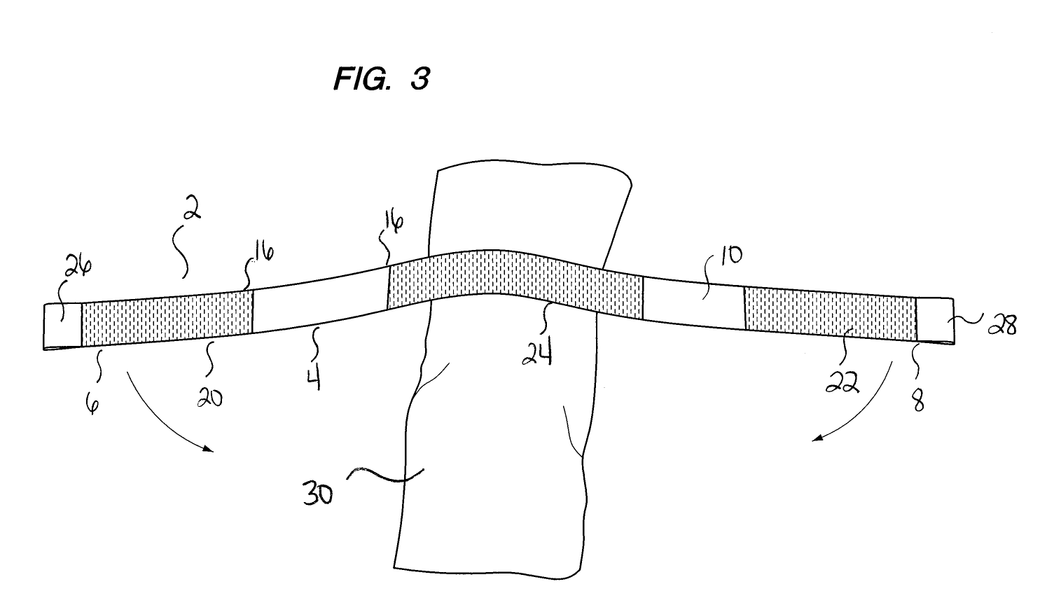

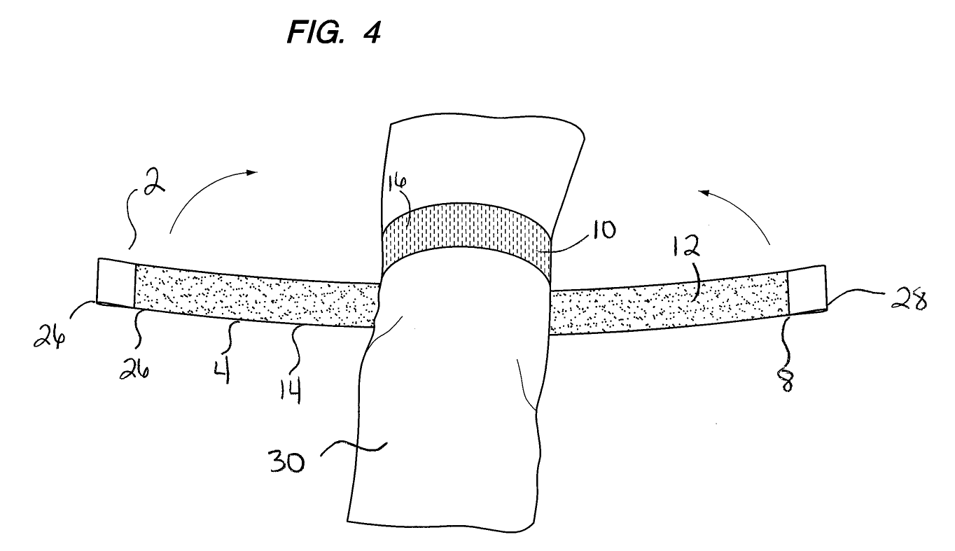

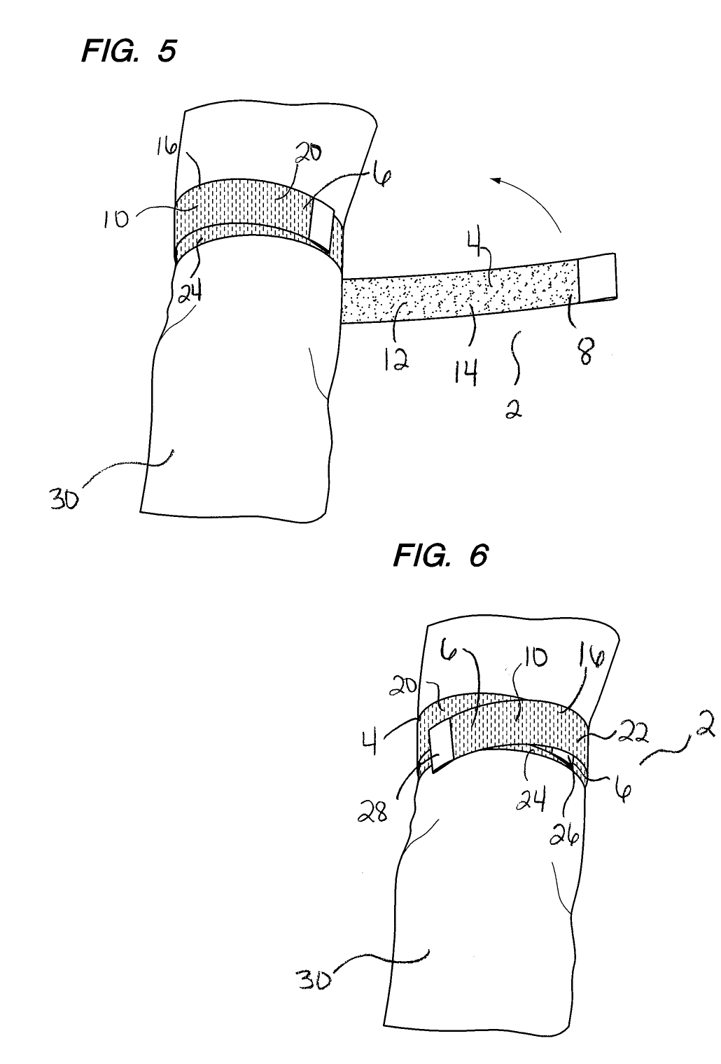

In reference to FIG. 3, in application the band 4 is placed across a limb, or appendage 30, the band 4 being adapted to encircle the appendage 30 bi-directionally. The band 4 is provided with a width designed to make application of the tourniquet 2 tolerable for a suitable period while the injured person is transported. The use of a wide elastic band does not pinch, while double stretch fabric, which pulls from opposite directions, means the tourniquet 2 can be applied very tightly, where necessary.

As the band 4 is extended from its first end 6 to encircle the appendage 30, the loop-type fastener means 14 at the second face 12 engages the hook-type fastener means 16 of the first face 10, which may be at the second end 8, where the appendage 30 has a large diameter, or, more typically, at the middle third 24 of the tourniquet 2.

Where the band 4 is first engaged by the loop-type fastener means 14 the second face 12 being secured to the hook-type fastener means 24 at the middle third, the tourniquet 2 is further extended from its second end 8 to encircle the appendage 30, and the loop-type fastener means of the second face 12 engages the hook-type fastener means of the first face 10 at the first end 6 previously attached over the hook material 24 at the middle portion. The applied pressure of the tourniquet 2 is increased with the additional hook and loop attachment where the application of the tourniquet 2 is done in this fashion.

The tourniquet 2 provides a readily available and easily applied, mechanism to decrease blood loss immediately after injury. The tourniquet 2 can be applied rapidly to slow blood loss due to vascular injury, and it is possible to apply the tourniquet 2 with one hand, even by an injured patient, particularly when the hook fastener material 16 and loop fastener material 14 is applied to the uniform. The tourniquet can be adjusted with one hand, as well, such as by changing a second end 8 while maintaining control of hemorrhage with the first end 6 that remains adherent to the hook-type fastener material 16. The tourniquet 2 can be applied with bi-directional force with or without use of a hook-type anchor already on the garment.

Rapid control of bleeding without limb-threatening ischemia is thus possible, such that were the tourniquet 2 supplied to every soldier or emergency personnel, it could result in decreased mortality, reduction in the need for transfusion (or the amount thereof), and finally, lessen the risk of amputation for field or emergency situations. In the event of trauma to an appendage, the tourniquet 2 can typically be applied within 30 seconds to control and decrease blood loss.

The simple band 4 allows various levels of control of bleeding, and without causing greater injury. The tourniquet 2 is light in weight, packs down to a very small package, and presents no additional risk from heat, such as the risk of flammability as is found in rubber tubing tourniquets. Thus, the tourniquet 2 can always be within easy reach and readily deployed.

The tourniquet 2 is also adjustable, in that it is as easy to secure as it is to release, so that quick adjustments can be made, such as for decreasing or increasing the level of tightness, and thus pressure. The ready and simple method of applying the tourniquet 2 permits various levels of pressure, partly due to the use of an elastic form of bandage 4, and as such the blood flow decrease may be partial to substantially complete. The method can be applied to decrease the distal pulse/blood flow a significant amount, by about 50, 60 or 70 percent, or more, as determined by Doppler flow measurements, though the tourniquet 2 should not, however, be so occlusive that ischemia results from total compression blood flow.

The tourniquet may also be permanently affixed to a garment at a permanent attachment point, such as by stapling, gluing and sewing the tourniquet at a desired or preferred location, such as a region on the uniform corresponding to an upper or lower extremity.

The tourniquet 2 can be supplied as part of emergency kits, particularly kits designed for vehicles, hiking, camping, household emergencies and the like.

A disc may be provided as part of such a kit, comprising a substantially cylindrical, hard, non-compressible material. The disc provides a surface for contacting the appendage 20, and connected to the band by having hook-type fastener applied to a surface opposite the surface contacting the appendage.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

I claim:

1. A tourniquet comprising:
a band comprised of an elastic and flexible material, said band having a width and a length, a first end and a second end, and a first face and second face,
wherein at least a portion of said first face comprises hook-type fastener means located within a third of said band at said first end, within a third of said band at said second end, and within a middle third of said band, and
wherein said second face comprises loop-type fastener means spanning the length of said second face.

2. The tourniquet of claim 1 whereby when said band is applied across an appendage said band has length sufficient to encircle said appendage bi-directionally,
wherein when said band is applied at said middle third across the appendage and extended from its first end to encircle said appendage said loop-type fastener means of the second face engages said hook-type fastener means of said first face at said middle third,
and wherein when said band is subsequently extended from its second end to encircle said appendage said loop-type fastener means of the second face at said second end engages said hook-type fastener means of said first face at said first end.

3. The tourniquet of claim 1 wherein said loop-type fastener means of said second face of said first end engages said hook-type fastener means of said middle third of said first face.

4. The tourniquet of claim 1 wherein said loop-type fastener means of said second face of said second end engages said hook-type fastener means of said middle third of said first face.

5. The tourniquet of claim 1 wherein said loop-type fastener means of said first face of said second end engages said hook-type fastener means of said second end of said first face.

6. The tourniquet of claim 1 further comprising a point of attachment for said tourniquet on a garment.

7. The tourniquet of claim 6 wherein said tourniquet is affixed by a method selected from the group consisting of stapling, gluing and sewing.

8. The tourniquet of claim 6 wherein said tourniquet is affixed to said garment at a region corresponding to an extremity.

9. The tourniquet of claim 6 wherein said tourniquet is affixed to said garment at a region corresponding to the neck or head region.

10. The tourniquet of claim 6 wherein said tourniquet is affixed to said garment at a region corresponding to the torso.

11. The tourniquet of claim 1 further comprising a disc secured to said tourniquet situated to apply focal pressure at a location of bleeding or sub-surface blood flow.

12. A method for impeding hemorrhage at a site on the body by applying a tourniquet to compress a vessel at a region to decrease bleeding and blood flow to tissue supplied below that region, said method comprising the steps of:
a) applying a tourniquet at said region, said tourniquet comprising a band comprised of an elastic and flexible material, said band having a width and a length, a first end and a second end, and a first face and second face, wherein at least a portion of said first face comprises hook-type fastener means located within a third of said band at said first end, within a third of said band at said second end, and within a middle third of said band, and wherein said second face comprises loop-type fastener means spanning the length of said second face, whereby said band is adapted to encircle said body part bi-directionally,
b) extending said band from its first end to encircle said body part, whereby said loop-type fastener means of the second face engage said hook-type fastener means of said first face, and
c) extending said band from its second end to encircle said body part, whereby said loop-type fastener means of the second face engage said hook-type fastener means of said first face, whereby blood flow is decreased to tissue below said region.

13. The method of claim 12 wherein said loop-type fastener means of said second face at said first end engages said hook-type fastener means of said first face at said second end.

14. The method of claim 12 wherein said loop-type fastener means of said second face at said first end engages said hook-type fastener means of said first face at said middle third.

15. The method of claim 14 wherein said loop-type fastener means of said second face at said second end subsequently engages said hook-type fastener means of said first face at said first end.

16. The method of claim 12 further comprising a point of attachment for said tourniquet on an article of clothing.

17. The method of claim 16 wherein said attachment point is provided by hook-type fastener material secured to said garment.

18. The method of claim 12 further comprising at least one disc secured to said tourniquet situated to apply focal pressure at a location of bleeding or sub-surface blood flow.

19. The method of claim 12 wherein said blood flow decrease is partial.

20. The method of claim 12 wherein said blood flow decrease is significant as assessed by Doppler flow measurement.

21. The method of claim 12 wherein said blood flow decrease is substantially complete.

22. The method of claim 12 wherein said tourniquet is applied at a region corresponding to an extremity.

23. The method of claim 12 wherein said tourniquet is applied at a region corresponding to the neck or head region.

24. The method of claim 12 wherein said tourniquet is applied at a region corresponding to the torso.
